(19) United States
(12) Patent Application Publication
(10) Pub. No.: US 2013/0331293 A1
(43) Pub. Date: Dec. 12, 2013

(54) AUTOMATED HIGH-CONTENT IMAGE ANALYSIS SYSTEM AND METHODS AND USES THEREOF

(71) Applicant: Rutgers, The State University of New Jersey, (US)

(72) Inventors: Martin L. Yarmush, Newton, MA (US); Tim Maguire, Piscataway, NJ (US); Nir I. Nativ, West Orange, NJ (US); Gabriel Adam Yarmush, Linden, NJ (US); Alvin I. Chen, Holmdel, NJ (US); Francois Berthiaume, Metuchen, NJ (US); David Xu Dong, South Plainfield, NJ (US)

(21) Appl. No.: 13/717,142

(22) Filed: Dec. 17, 2012

Related U.S. Application Data
(60) Provisional application No. 61/576,563, filed on Dec. 16, 2011.

(51) Int. Cl. G01N 33/92

(52) U.S. Cl. CPC G01N 33/92
USPC 506/10; 435/29; 382/133

(57) ABSTRACT

This invention relates to algorithms, methods and products useful in assessing steatosis level of tissues, using an automated high-content image analysis framework. The algorithms, methods and products are particularly useful in liver transplantation by providing a fast, precise and reproducible steatosis level estimation pre-transplantation. The invention also enables in vitro high throughput screening of drug candidates for reducing intra-cellular triglyceride content in the form of lipid droplets from fatty livers.

### CROSS REFERENCE TO RELATED APPLICATIONS

This application claims priority under 35 U.S.C. §119(e) to U.S. Provisional Patent Application Serial No. 61/576,563, filed on Dec. 16, 2011, which is hereby incorporated by reference in its entirety.

### STATEMENT OF FEDERALLY SPONSORED RESEARCH

This invention was made at least in part with government support under NIH Grant No. R01DK059766 and NIH-funded Biotechnology Training Fellowship. The United States government has certain rights in the invention.

### FIELD OF THE INVENTION

The present invention relates to the field of liver transplantation by providing a product and methods for assessing steatosis state of liver tissues using image analysis. The method is amenable to in vitro high throughput screening of agents for their potential in reducing intra-cellular triglyceride content in the form of lipid droplets from fatty livers and thus improving liver transplantation outcome.

### BACKGROUND OF THE INVENTION

During the last decade, there has been a yearly shortage of about 10,000 livers for orthotropic transplantation, which results in approximately 2,000 deaths per year of patients awaiting liver transplantations in the U.S. About 1,000 livers offered for orthotropic transplantation are discarded yearly due to abnormally high hepatic intra-cellular triglyceride level content, known as steatosis. Moderate to severe steatosis is a risk factor for primary graft non-function due to increased sensitivity to ischemia reperfusion injury introduced during liver harvesting and transplantation.

Orthotopic liver transplantation is severely limited by donor scarcity. This has motivated the development of strategies to recover livers from deceased donors currently not considered suitable for transplantation. Macrosteatosis, defined as the accumulation of triglycerides (TG) in the form of large lipid droplets that displace the nucleus to the cell periphery, when found in more than 30% of the hepatocytes, is a very common cause of donor ineligibility. Such livers are more sensitive to ischemia/reperfusion (I/R) injury inherent to liver transplantation, and more prone to primary non-function, as well as increased morbidity and mortality post-transplantation. The incidence of hepatic macrosteatosis is likely to surge due to the obesity epidemic. Thus, techniques to salvage macrosteatotic livers could significantly enhance donor supply in both the short and long terms.

A variety of approaches targeting some of the downstream effects of macrosteatosis during I/R have shown promise in pre-clinical and clinical settings. However, several studies suggest that excessive hepatic lipid storage is a primary cause of the exuberant I/R response, especially when in the macrosteatotic form. Therefore, an alternative approach could be to eliminate the hepatic intracellular lipid droplets, thus decreasing the frequency of macrosteatotic hepatocytes below acceptable levels. Dieting, exercise, and fibrate drugs over several weeks have been shown to decrease macrosteatosis and enable living donor liver transplantation. In a rat liver model of macrosteatosis (induced by a choline and methionine-deficient diet; CMDD), switching to a normal diet 3 days prior to transplantation reduced intrahepatic TG content by 35% and increased recipient viability from 0% to >75% post-transplantation (Nativ et al. Am. J. Transplant, 2012, 12, 3176-3183).

Diet/drug-induced macrosteatosis reduction occurs over days to weeks, a timescale that is not applicable to deceased human donors, which would require macrosteatotic reduction ex vivo within a few hours. Animal studies have demonstrated the feasibility of macrosteatosis reduction via machine perfusion of explanted steatotic livers. This process was accelerated by introducing agents that promote lipid metabolism. However, a thorough exploration of these agents, or combinations thereof, has yet to be performed. Furthermore, there has been little investigation of the impact of accelerated macrosteatosis reduction on the viability and function of hepatocytes, parameters that are critical for the successful outcome of liver transplantation (Nativ et al. Am. J. Transplant., 2012, 12, 3176-3183).

A suitable cell culture model would facilitate the evaluation of these agents with the ultimate goal of developing protocols to promote accelerated macrosteatosis reduction and functional recovery of transplanted steatotic livers. Microsteatotic hepatocyte culture systems have been described in this context in the literature (e.g., Berthiaume et al., J. Surg. Res., 2009, 152:54-60); however, their relevance is unclear given that clinical evidence suggests that macrosteatotic—and not microsteatotic—livers are hypersensitive to I/R. Herein, we describe a novel macrosteatotic hepatocyte culture system to investigate the effect of macrosteatosis on viability and liver-specific functions in hepatocytes. We also use this system to explore the impact of accelerated macrosteatosis reduction rate on viability and the recovery of such functions.

The current method of assessing the pathological state of the tissue is based on the pathologist's “naked eye” analysis, which lacks precision and reproducibility in determining the clinically relevant cutoff of macrosteatosis and may overlook various features and trends in the tissue steatosis state, such as total surface area of fat droplets which may represent the total triglycerides stored as fat vacuoles in the tissue. (El-Badry et al. Annals of Surgery, 2009, 250, 691-697; Fiorentino et al. Liver Transplant, 2009, 15, 1821-1825; and Heller et al. J. Clin. Med. Res., 2011, 191-194). Therefore, a more precise, reproducible method for assessing pathological state of a tissue, such as liver tissue, for possible steatosis is needed.

### SUMMARY OF THE INVENTION

The present invention provides products and methods to meet the foregoing needs. Specifically, the present invention provides an automated high content image analysis framework for assessing the intracellular fat content in the form of lipid droplets of pathology tissue sections and in vitro culture systems, and methods and uses thereof.

In one aspect, the present invention provides a method for assessing the state of steatosis of a tissue such as liver obtained by biopsy or tissue in an in vitro culture system, the method comprising: (a) providing the tissue as mentioned above; (b) conducting an automated image analysis on a digital image of the sample tissue or cells thereof; (c) generating an array of parameters related to steatosis; and determining intra-cellular triglyceride content in the form of lipid droplets (steatosis) in the tissue based upon the parameters. The parameters are independently selected front fat droplets count, total fat droplet cross-sectional area, average fat droplet area, average fat droplet equivalent diameter, total area percent steatosis, nuclei count, total nuclei area, average nucleus area, nuclei proximity to the lipid droplets and average fat area per nucleus, or the like.

In another aspect, the present invention provides an automated high-content image analysis system for assessing intracellular fat content of a tissue, comprising: (a) a software program comprising a plurality of object-oriented module-based algorithms capable of segmenting intracellular fat droplets, cell nuclei, sinusoidal spaces, erythrocytes, and other cellular structures; (b) a clustering mechanism capable of roughly separating fat, nuclei, and surrounding tissue before more sensitive, model-driven approaches to tune the classification schemes toward maximal accuracy; (c) an algorithm capable of generating Edge characteristics using the Laplacian of Gaussians technique; (d) an active contour or level set algorithm; and (e) a graph partitioning mechanism.

In another aspect, the present invention provides a computer readable medium having instructions for enabling a computer system to implement an automated high-content image analysis of a tissue.

In another aspect, the present invention provides a method of identifying agents for reducing triglyceride content in the form of lipid droplets in a liver in need of such reduction, comprising: (1) obtaining a tissue sample from a fatty liver or lean liver and then induced for steatosis in vitro; (2) contacting said tissue sample with one or more candidate agents; and (3) subjecting said tissue sample to an automated image analysis system to determine the state of steatosis in the sample, wherein a reduction in the state of steatosis in the sample contacted with a candidate agent relative to an untreated control sample is indicative of the ability of the candidate agent to reduce the triglyceride content in a fatty liver.

In another aspect, the present invention provides a device for assessing intra-cellular triglyceride content in the form of lipid droplets in a liver tissue for the state of steatosis, comprising, an automated high-content image analysis system.

In another aspect, the present invention provides a machine learning framework for determining the sensitivity and specificity of the automated high-content image analysis system in comparison with analysis by a clinician, comprising bootstrapping, rooted trees, Bayesian clustering, graphical partitioning, and a plurality of forms of component analysis (PCA, ICA, etc.), wherein the methods provide a way for the program to automatedly learn based on a small portion of the full dataset that is used as a training set. The machine learning framework program is able to a) determine the inputs that maximize accuracy and stability; and b) reduce the number of inputs that the clinician needs to define, thereby becoming more automated given more data.

In another aspect, the present invention provides an automated classifier for segregating macrosteatotic fat droplets from microsteatotic fat droplets, based on the propensity of a given sized droplet to cause a nucleus displacement toward the side of a cell, wherein macrosteatotic droplets cause a positional shift to the cell nucleus, pushing them toward the side of the cell, wherein the nuclei remain in a state such that they are directly adjacent to the macrosteatotic droplet.

In another aspect, the invention also provides a software (Matlab code) program containing a graphic user interface (GUI), which enables: 1) upload of digital images, 2) their high-content image analysis, 3) display of the results in the forms of images, tables and histograms, 4) output of the analysis results to external formats, and 5) prediction of state of liver steatosis based on machine learning.

These and other aspects of the present invention will be better appreciated by reference to the following drawings and detailed description.

### BRIEF DESCRIPTION OF THE DRAWINGS

FIG. 1 illustrates a flow chart of image processing and classification. A. Original histology image; B. Edge function of the color gradient of the original image; C. Seedpoints for lipids and nuclei determined from local intensity maxima and minima, respectively; D. Region-growing routine iteratively maximizes region scores based on morphology, boundary strength, and intensity; E. Final segmented image showing lipid droplets, cell nuclei, nonparenchymal cells, and sinusoidal spaces.

FIG. 2 illustrates a process to generate steatosis related parameters from image analysis of liver phatology H&E stained slide.

FIGS. 3A-C illustrates a process to generate steatosis related parameters from image analysis of in vitro cultured hepatocytes. A. High throughput image analysis based software interface. B. Averaged droplet size in lean, micro steatosis and macro steatosis primary hepatocytes as quantified by the software. C. Number of macro steatosis droplets per nuclei in lean, micro and macro steatosis primary hepatocytes as quantified by the software (macro steatotic droplets are based on the minimal cross-sectional surface area of droplets which can dislocate the nuclei, about 350 μm).

FIG. 4 illustrates Lipid droplet minimal cross-sectional surface area defining macrosteatosis. Lipid droplet sizes were measured within hepatocyte cultures exposed to steatosis inducing medium for 6 days. A survey of individual cells revealed that nuclear dislocation to the cell periphery occurred when the cross-sectional surface area of lipid droplets reached 350 μm². Shown is a representative image of hepatocyte cultures showing lipid droplet sizes and nuclear location. Bar=50 μm.

FIG. 5 illustrates the ability of the product to quantify the reduction in steatosis level of hepatocytes in vitro culture while illustrating hepatocyte morphology and lipid content during macrosteatosis reduction. Macrosteatotic hepatocytes cultures were supplemented (SRS) or not (NSRS) with steatosis reducing agents for 2 days. A. Bright-field (top) and fluorescent images of Nile red and Hoechst stained macrosteatotic hepatocytes (bottom) after 2 days of culture. B, C. Macrosteatosis droplet number>350 μm² and Intracellular TG content as a function of steatosis reduction media. Data=±S.E.N=6. ·p<0.01 vs. D13 Lean. ⁰p<0.03 vs. D11 Fat. Bar=50 μm.

FIG. 6 illustrates human H&E stained liver pathology slides segmented for lipid droplets as well as histograms of lipid droplets count vs. size of the lipid droplets based on image analysis results.

FIG. 7 illustrates high-content image analysis and fat size distribution in a Zucker rat liver tissue analyzed.

FIG. 8 illustrates quantified metrics from a liver tissue analyzed in FIG. 7.

FIG. 9 illustrates the ability of the product to quantify the nuclei proximity to a lipid droplet.

FIG. 10 illustrates the ability of an automated classifier, wherein the software linearly combines the nucleus adjacency score and the size score to form a latent score that classifies macro and micro steatosis.

### DETAILED DESCRIPTION OF THE INVENTION

Cell culture and animal data suggest that decreasing hepatocyte intracellular triglyceride content reduces hepatocyte sensitivity to hypoxia-reoxygenation in vitro, and increases liver graft function in vivo. Screening a large number of metabolically active agents that promote the rapid reduction of hepatic intra-cellular triglycerides, and applying them to explanted livers in an ex vivo perfusion system to “defat” livers, may enable transplantation of otherwise discarded liver grafts (Nativ et al. Am. J. Transplant., 2012, 12, 3176-3183).

In order to assess the potency of these agents in reducing the intracellular triglyceride content of liver cells or to predict the sensitivity of the cells or liver grafts to ischemia reperfusion injury based on tissue specific visual markers, two techniques are being used: 1) H&E stained liver biopsy slide (frozen or paraffin embedded); and 2) in vitro model of hepatocytes incubated with free fatty acids to achieve various levels of steatosis, including clinically relevant macro-steatosis. Various pathological tissue mapping techniques are disclosed in, for example, U.S. Pat. No. 7,483,554 to Kotsianti et al., which is hereby incorporated by reference.

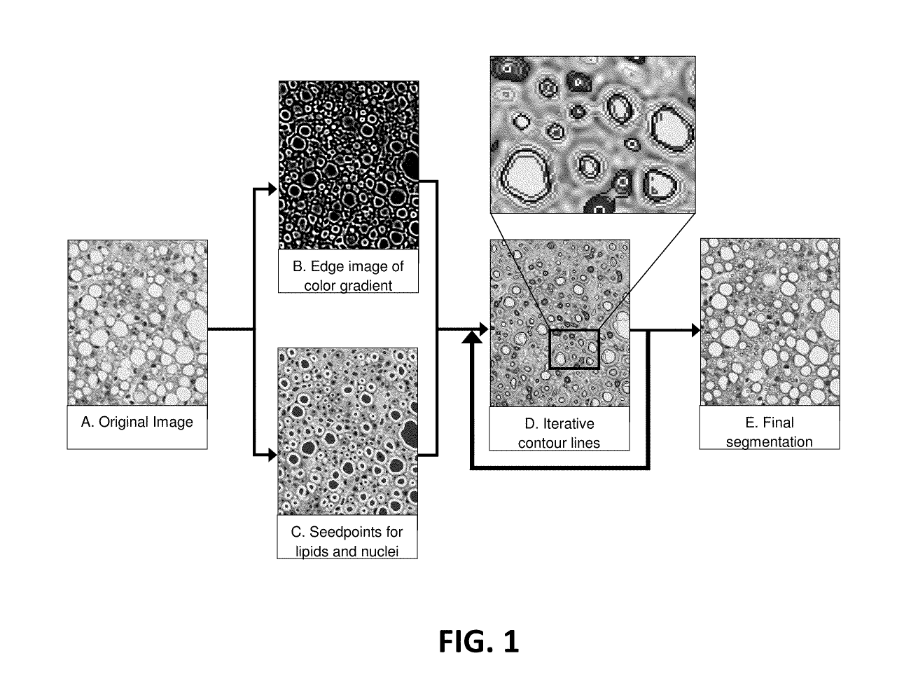

The present invention provides an automated high content image analysis framework for assessing the intracellular fat content in the form of lipid droplets of pathology H&E stained tissue sections and in vitro culture systems, and methods and uses thereof. A flow chart of image processing and classification is shown in FIG. 1.

The high content image analysis framework contains series of object-oriented module-based algorithms to robustly segment intracellular fat droplets, cell nuclei, sinusoidal spaces, erythrocytes, and other cellular structures. In one mode of the disclosed invention, a multi-threshold approach is combined with shape, intensity, texture, and edge based descriptors to accurately differentiate between the many different components of tissue. Clustering techniques such as K-means clustering is employed initially in all embodiments to roughly separate fat, nuclei, and surrounding tissue before more sensitive, model-driven approaches tune the classification schemes toward maximal accuracy. Edge characteristics are imparted to the system using the Laplacian of Gaussians technique, which highlights steep changes in intensity characteristic of object boundaries. The Watershed filter is another common technique, used here to refine the geometrical segmentation criteria.

In another mode, the above framework will be further augmented with active contour or level set algorithms, with the central assumption being that the structures of interest have a repetitive forum of geometry which can be exploited by seeking a probabilistic model that explains the variation of the shape and imposes a priori constraints on the segmentation routine. Specifically, the geometries and intensities of the structures are known, as are the expected form and degree of deviation. Graph partitioning is employed as a final component during the multithreshold and level set routines as an overarching framework to track the progression of geometrical change in response to changing thresholds and sets. In the case of multithreshold approach, as the stringency of the highpass filter is iteratively increased, the number of structures which pass the threshold will decrease, and will subserve those structures which passed the previous threshold. Weighted directed graphs are used to track these relationships.

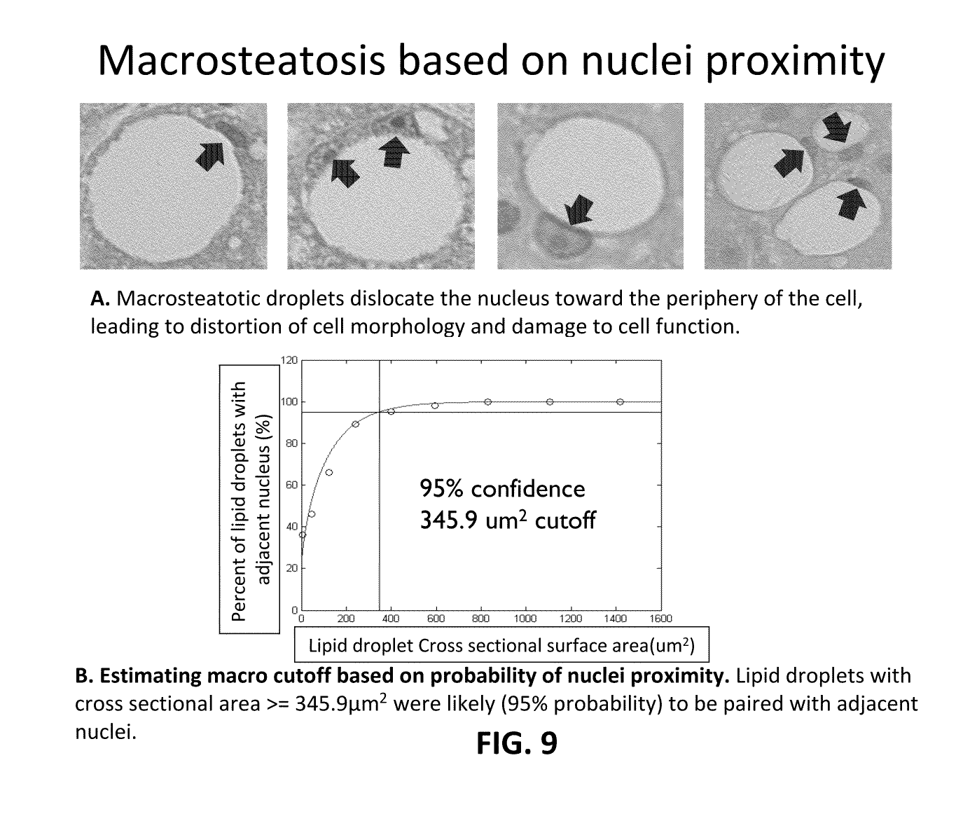

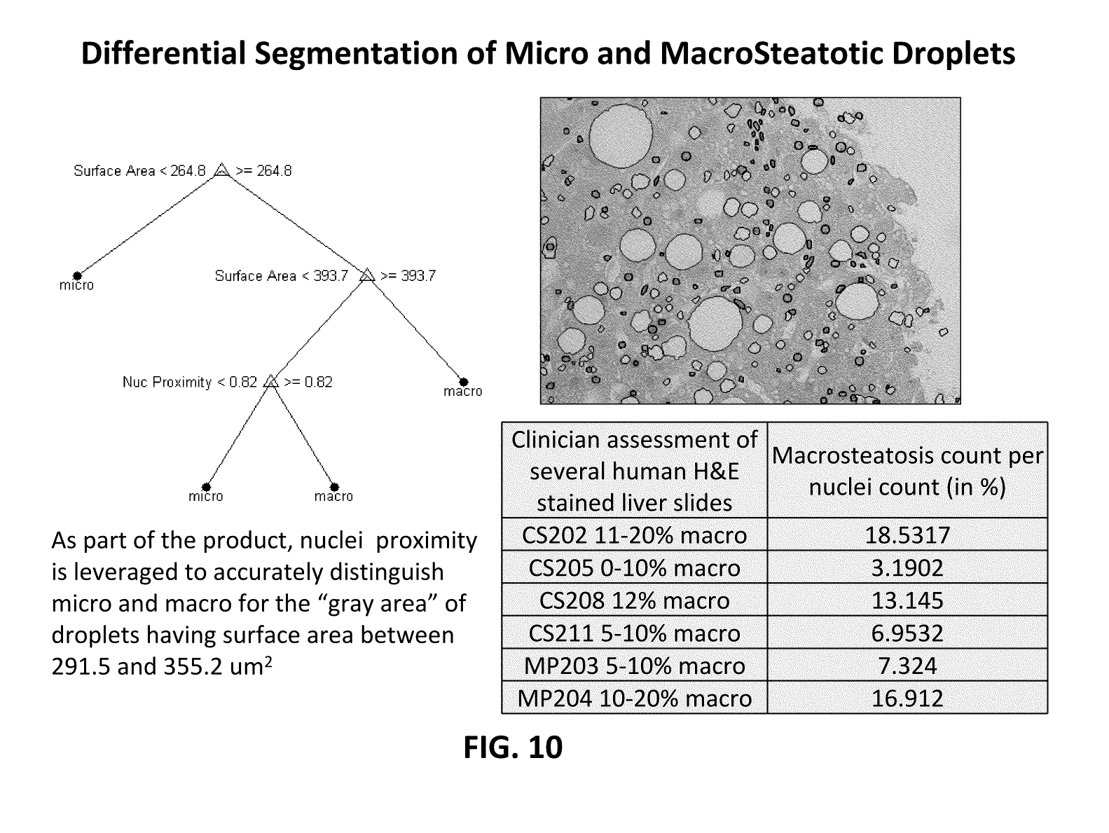

The present invention also describes an automated classifier to segregate macrosteatotic fat droplets from microsteatotic fat droplets based on the propensity of a given sized droplet to cause a nucleus displacement toward the side of the cell. Rather than to arbitrarily define a size-based cutoff above which all droplets classify as macrosteatotic, the system imparts the observation that macrosteatotic droplets tend to cause a positional shift to the cell nucleus, specifically pushing them toward the side of the cell. A direct consequence of this is that the nuclei then remain in a state such that they are directly adjacent to the macrosteatotic droplet. Microsteatotic droplets are too small to have this effect. In one mode of the invention, the software uses a “nucleus adjacency” probability metric to define a size-based cutoff that categorizes macro and micro droplets. In another mode, the software linearly combines the nucleus adjacency score and the size score to form a latent score that classifies macro and micro steatosis (FIGS. 9-10)

Also disclosed is an algorithm that automatedly determines the level of steatosis of any given tissue slice or culture system. Several methods of defining steatosis levels are incorporated and the user can select the preferred modality. These include a metric which finds the ratio of total fat area versus total tissue area, or similarly, total macrosteatotic area versus total tissue area. A second method of determining steatosis level is by using counts rather than areas—this method is more in line with the clinician approach. Namely, steatosis is defined as the number of macrosteatotic droplets in the slice divided by the number of cell nuclei in the slice. A third mode combines the macrosteatotic area metric with the macro count metric and uses a latent classification metric to determine percent steatosis in the tissue.

The present invention also provides a machine learning framework that can determine the sensitivity and specificity of the analysis in comparison to the clinician, and adjust parameters to improve accuracy and stability. These learning methods include bootstrapping, rooted trees, Bayesian clustering, graphical partitioning, and various forms of component analysis (PCA, ICA, etc). The goal of these methods is to provide a way for the program to automatedly learn based on a small portion of the full dataset that is used as a training set. In this way, the program is able to a) determine the inputs that maximize accuracy and stability; and b) reduce the number of inputs that the clinician needs to define, thereby becoming more automated given more data.

In addition, the invention utilizes a graphical user interface that allows for uploading of image files of multiple tissue types and magnifications, adjustment of settings, manual segmentation and correction, batch processing, graphical displays, and outputs to numerous formats.

Thus, in one aspect the present invention provides a product that incorporates a software program (Matlab code) including a graphic user interface (GUI) and enables: 1) the upload of digital images, 2) their high content image analysis, 3) display of the results in the forms of images, tables and histograms, 4) output of the analysis results to external formats, and 5) prediction based on machine learning.

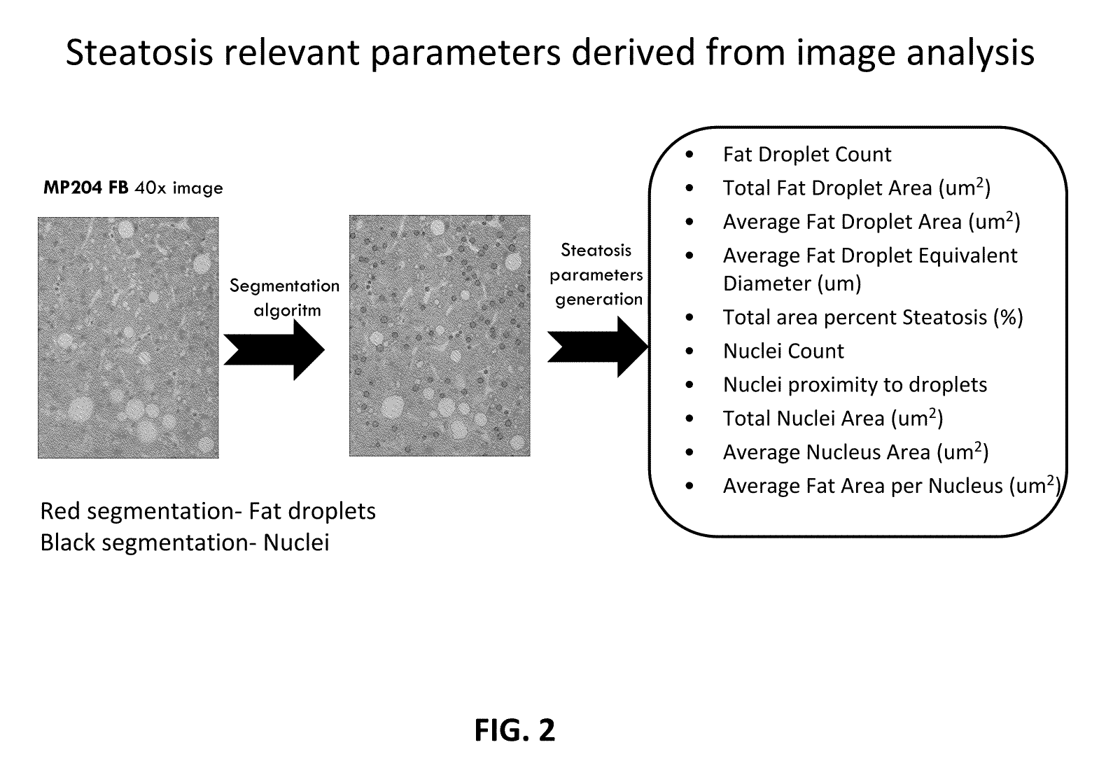

The product of the present invention allows for high throughput, automated, quantifiable image analysis of relevant parameters for assessment of tissue pathology (in vivo) as well as tissue cultures (in vitro) to yield fast, precise and reproducible analysis of the tissue pathological steatosis state. This steatotic state serves as a predictor for the success of the liver graft transplantation. The product enables precise and reproducible results as well as generation of an array of parameters which cannot be quantified by the naked eye. The image analysis software can segment the fat droplets on H&E stained liver pathology slides with high specificity and sensitivity. Quantifiable metrices can be generated to represent liver steatosis. The steatosis relevant parameters may include, but are not limited to, fat droplet count, total fat droplet area, average flit droplet area, average fat droplet equivalent diameter, total area percent steatosis, nuclei count, average nucleus area, nuclei proximity, and average fat area per nucleus, or the like (see FIG. 2). In addition, these parameters may better indicate the state of the tissue than a single parameter, such as the percentage of cells in the tissue which contain one or more fat vacuoles, known as percent steatosis. The array of steatosis relevant parameters generated by the product may be classified using liver graft function post-transplantation clinical data as a training set to yield steatosis state specific image based signature. This signature enables prediction of the state of steatotic liver tissue as well as the outcome of the transplantation.

In another aspect the invention provide methods of assessing state of liver steatosis using the product, and methods for predicting the success of liver graft transplantation.

The product assists liver transplantation healthcare professionals in assessment of the pathologic state of donor liver steatosis based on high content image analysis of steatotic relevant parameters. Donor livers that would have been discarded based on the steatosis criteria for transplantation can thus be successfully transplanted after evaluation with the present product and methods.

In addition, the product contributes to the field of liver transplantation as part of an in vitro high throughput chug screening apparatus used to assess the potential of various agents to reduce the percentage of macrosteatotic hepatocytes and/or to reduce intra-cellular triglyceride content from fatty liver pre-transplantation and thus to improve transplantation outcome. In this embodiment, the invention provides a method of identifying agents capable of reducing triglyceride content in a liver in need of such reduction comprising obtaining a tissue sample from a fatty liver; contacting said sample with a potential agent; and subjecting said sample to an automated image analysis as described hereinabove to determine the state of steatosis in the sample, wherein reduction in the state of steatosis in a sample contacted with the potential agent relative to an untreated control sample is indicative of an agent capable of reducing triglyceride content in a fatty liver.

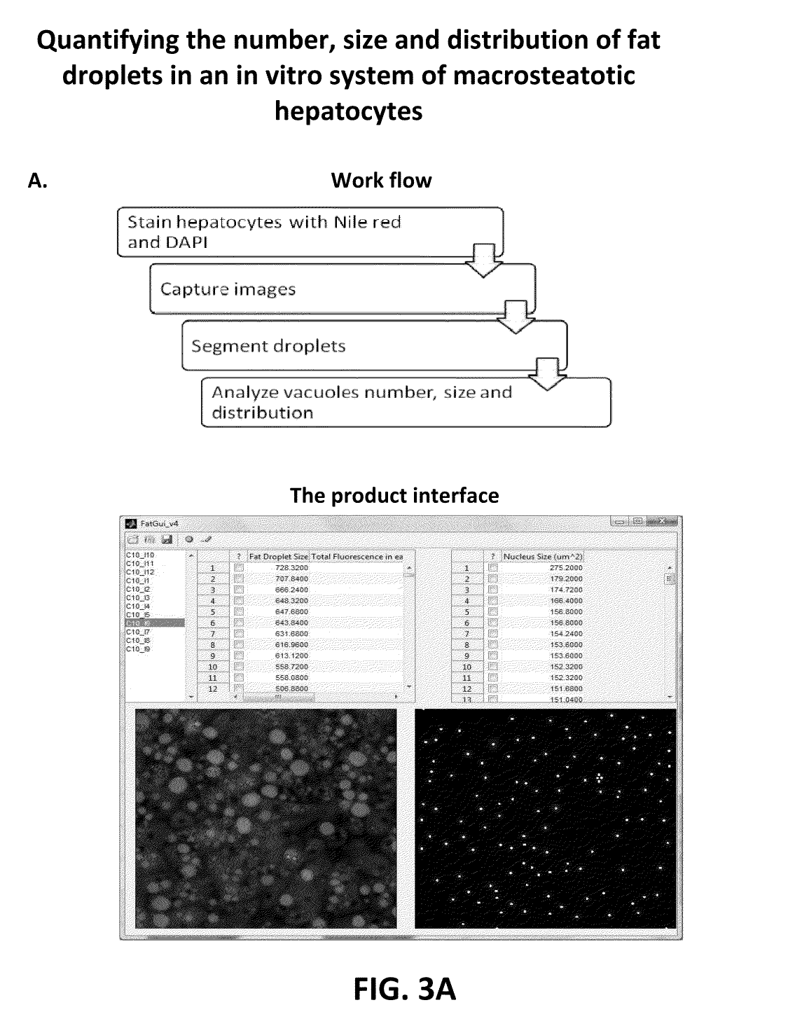

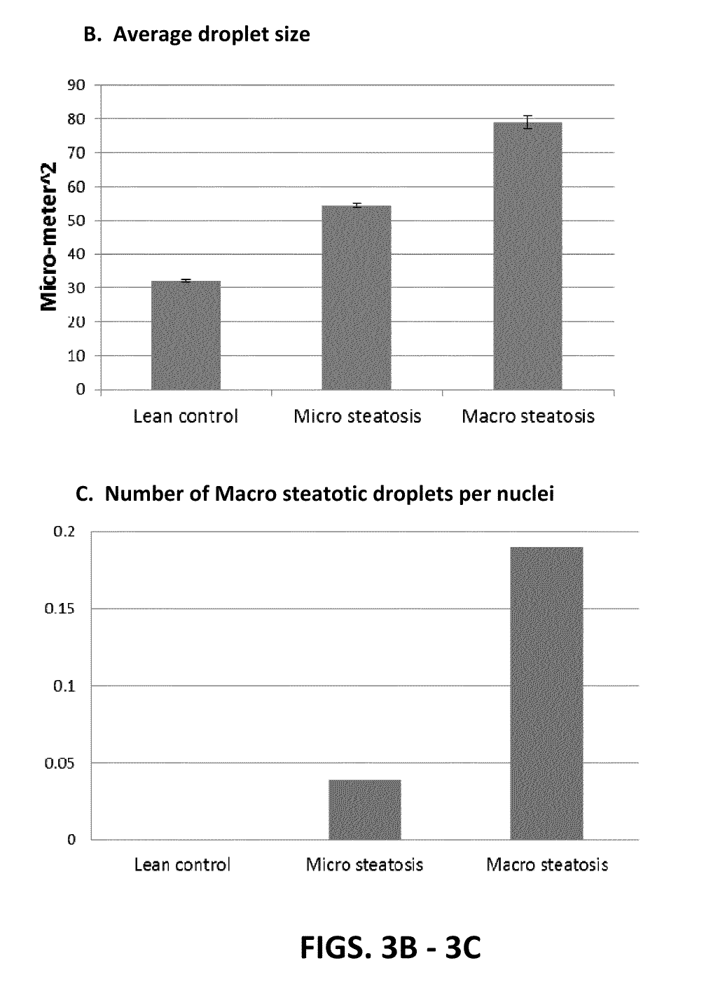

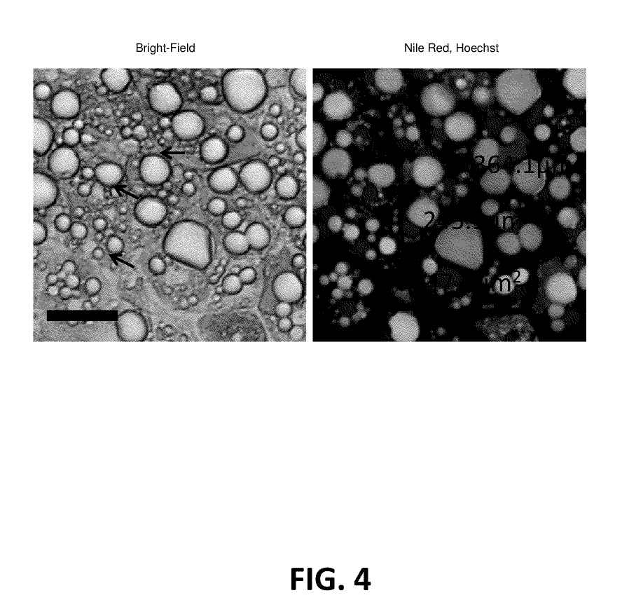

In this embodiment, the triglyceride content may first be visually determined by epifluorescent microscopy while using triaglyceride specific stain, for example, Nile-red. Digital images of the tissue are analyzed for an array of relevant parameters in a fast, precise and reproducible manner (FIGS. 3-5). This allows for a more intense analysis than conventional methods for determining the effect of the potential agent on the tissue. Furthermore, the classification algorithm selects the most important parameters and utilizes them to construct the classifier. This allows the algorithm to learn from itself and to improve its accuracy as more experimental data are generated.

In one embodiment of this aspect, the present invention provides a method for assessing the state of steatosis of a liver, the method comprising: (a) providing a sample tissue of the liver; (b) conducting an automated image analysis on a digital image of the sample tissue or cells thereof; (c) generating an array of parameters related to liver steatosis; and determining intra-cellular triglyceride content in the form of lipid droplets in the liver tissue based upon the parameters.

In another embodiment of this aspect, the method contains the steps of: 1) uploading digital images, 2) analyzing high content images, 3) displaying results in the forms of images, tables or histograms, 4) outputting the analysis results to external formats, and 5) predicting state of liver steatosis based on machine learning.

In another embodiment of this aspect, the array of parameters are independently selected from fat drop count, total fat droplet area, average fat droplet area, average fat droplet equivalent diameter, total area percent steatosis, nuclei count, total nuclei area, average nucleus area, nuclei proximity to a lipid droplet, and average fat area per nucleus, or the like.

In another embodiment of this aspect, the array of parameters are independently selected from the group consisting of fat droplet count, total fat droplet cross-sectional surface area, average fat droplet cross-sectional surface area, average lipid droplet equivalent diameter, total area percent steatosis, nuclei count, total nuclei area, average nucleus area, nuclei proximity to a lipid droplet, and average fat area per nucleus.

In another aspect, the present invention provides an automated high-content image analysis system for assessing the intracellular fat content of pathology tissue sections and in vitro culture systems, comprising: (a) a software program comprising a plurality of object-oriented module-based algorithms capable of segmenting intracellular fat droplets, cell nuclei, sinusoidal spaces, erythrocytes, and other cellular structures; (b) a clustering mechanism capable of roughly separating fat, nuclei, and surrounding tissue before more sensitive, model-driven approaches to tune the classification schemes toward maximal accuracy; (c) Edge characteristics using the Laplacian of Gaussians technique; (d) an active contour or level set algorithm; and (e) a graph partitioning mechanism.

In one embodiment of this aspect, the automated high-content image analysis system further comprises a multi-threshold approach in combination with shape, intensity, texture, and edge based descriptors to accurately differentiate between different components of tissue.

In another embodiment of this aspect, the clustering technique is K-means clustering.

In another embodiment of this aspect, the Edge characteristics highlight steep changes in intensity characteristic of object boundaries and a Watershed filter to refine the geometrical segmentation criteria.

In another embodiment of this aspect, the structures of interest produced by said active contour or level set algorithm comprise a repetitive form of geometry that can be exploited by seeking a probabilistic model that explains the variation of the shape and imposes a priori constraints on the segmentation routine.

In another embodiment of this aspect, the graph partitioning is employed during the multithreshold and level set routines as an overarching framework to track the progression of geometrical change in response to changing thresholds and sets.

In another embodiment of this aspect, in the case of said multithreshold approach, as the stringency of the highpass filter is iteratively increased, the number of structures which pass the threshold will decrease, and will subserve those structures which passed the previous threshold, and wherein weighted directed graphs are used to track these relationships.

In another aspect, the present invention provides a computer readable medium having instructions for enabling a computer system to implement the method for assessing the state of steatosis of a liver as described above.

In another aspect, the present invention provides a method of identifying agents for reducing triglyceride content in the form of lipid droplets in a liver in need of such reduction, comprising: (1) obtaining a tissue sample from a fifty liver; (2) contacting said tissue sample with one or more candidate agent; and (3) subjecting said tissue sample to an automated image analysis to determine the state of steatosis in the sample, wherein a reduction in the state of steatosis in the sample contacted with the potential agent relative to an untreated control sample is indicative of the ability of the agent to reduce the triglyceride content in a fatty liver.

In another aspect, the present invention provides a device for assessing intra-cellular triglyceride content in the form of lipid droplets in a liver tissue for the state of steatosis, comprising an automated high-content image analysis system as described above.

In another aspect, the present invention provides an automated classifier for segregating macrosteatotic fat droplets from microsteatotic fat droplets, based on the propensity of a given sized droplet to cause a nucleus displacement toward the side of a cell, which imparts the observation that macrosteatotic droplets tend to cause a positional shift to the cell nucleus, specifically pushing them toward the side of the cell, rather than arbitrarily define a size-based cutoff above which all droplets classify as macrosteatotic, wherein the nuclei remain in a state such that they are directly adjacent to the macrosteatotic droplet.

In one embodiment of this aspect, the automated classifier comprises a software that uses a “nucleus adjacency” probability metric to define a size-based cutoff that categorizes macro and micro droplets.

In another embodiment of this aspect, the software of the automated classifier linearly combines the nucleus adjacency score and the size score to form a latent score that classifies macro and micro steatosis.

In another aspect, the present invention provides a method of determining steatosis level, comprising using an algorithm that automatedly determines the level of steatosis of any given tissue slice or culture system, and incorporating a plurality of methods of defining steatosis levels from which a user can select the preferred modality.

In another embodiment of this aspect, the method further comprises determining a metric that finds the ratio of total fat area to total tissue area, or total macrosteatotic area to total tissue area.

In another embodiment of this aspect, using counts rather than areas to define the level of steatosis, the method comprises defining steatosis as the number of macrosteatotic droplets in the slice divided by the number of cell nuclei in the slice.

In another embodiment of this aspect, the method combines the macrosteatotic area metric with the macro count metric and uses a latent classification metric to determine percent steatosis in the tissue.

In another aspect, the present invention provides a machine learning framework for determining the sensitivity and specificity of the analysis in comparison to a clinician, and adjusting parameters to improve accuracy and stability, comprising bootstrapping, rooted trees, Bayesian clustering, graphical partitioning, and a plurality of forms of component analysis (PCA, ICA, etc.), wherein the methods provide a way for the program to automatedly learn based on a small portion of the full dataset that is used as a training set.

In one embodiment of this aspect, wherein the program is able to a) determine the inputs that maximize accuracy and stability; and b) reduce the number of inputs that the clinician needs to define, thereby becoming more automated given more data.

In another aspect, the present invention provides a graphical user interface that allows for uploading of image files of multiple tissue types and magnifications, adjustment of settings, manual segmentation and correction, batch processing, graphical displays, and outputs to numerous formats.

The product and methods described herein serve as an assistance tool for pathologists when assessing steatotic liver for transplantation. The product allows the pathologist to quantify the level of liver steatosis in an accurate, reproducible manner with no variability among pathologists. In addition, this product and method reduce costs when used in high throughput screening for agents to reduce the intra-cellular triglyceride level of liver cells.

The product and methods of the invention improve the diagnosis of steatotic livers for successful transplantation and may reduce the incidence of unsuccessful transplantation of steatotic liver which requires an immediate and expensive additional liver transplantation.

The product and methods of the invention are also useful for automated high throughput analysis of intra-cellular fat droplets in an in vitro setting of adipocytes and hepatocytes for the identification of agents useful in the treatment of disorders including for example diabetes, non-alcoholic fatty liver disease, and alcoholic fatty liver disease.

In this application all terms unless otherwise defined take the ordinary meaning known to a person of ordinary skill in the art.

The following non-limiting example serves to further illustrate the present invention.

### EXAMPLE

### Experimental Procedures

### Hepatocyte Isolation and Culture

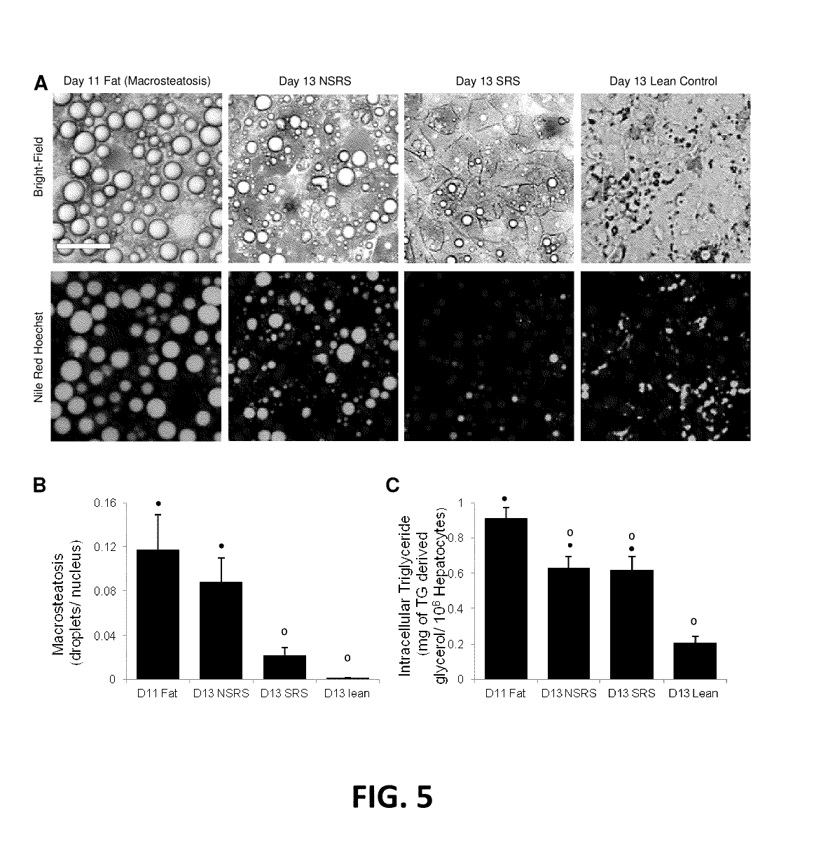

Male lean Zucker rats, (Charles River, Wilmington, Mass.) (310±20 g) were housed in a 12 h light-dark cycle and temperature-controlled environment (25° C.) with water and standard chow ad libitum. All experimental procedures were in accordance with National Research Council guidelines and approved by the Rutgers University Animal Care and Facilities Committee. Hepatocytes were isolated using a two-step in situ collagenase perfusion technique. Viability was 90±4% as determined by trypan-blue exclusion. Six-well culture plates (Beckton-Dickinson, Franklin Lakes, N.J.) were pretreated with 50 ug/ml rat type 1 collagen solution (Beckton-Dickinson) in 0.02M acetic acid (Sigma-Aldrich, St. Louis, Mo.) overnight at 4° C. and washed with phosphate buffered saline (PBS, Invitrogen, Grand Island, N.Y.). Freshly isolated hepatocytes were suspended (10⁶ cells/ml) in standard hepatocyte medium and seeded (10⁶ cells/well). After incubating the cells at 37° C. in a 90% air/10% CO₂ atmosphere for 24 h, medium was removed and a collagen gelling solution (0.5 ml/well) was added to form a gel overlay. Cultures were maintained in standard hepatocyte medium for 4 days with a fresh medium change every other day. Spent medium was collected (FIG. 5A, experimental days 1-5) for analysis.

### Steatosis Induction and Reversal

Five-day hepatocyte cultures were switched to steatosis-inducing medium. Standard hepatocyte medium was supplemented with 2 mM oleic acid, 2 mM linoleic acid, and 4% (weight to volume) bovine serum albumin (Sigma-Aldrich) for 3 days. Medium was replaced with fresh steatosis-inducing medium for another 3 days of steatosis induction and the spent medium was collected (FIG. 5A, experimental days 5-11). Following 6-day steatosis induction (11 days post-seeding), the medium was replaced with fresh hepatocyte medium with no steatosis reduction supplements (NSRS), or with a combination of the following steatosis reduction supplements (SRS): 10 μM forskolin, 1 μM GW7647, 10 μM scoparone (Sigma-Aldrich). 1 μM GW501516, 10 μM hypericin (Enzo, Faimingdale, N.Y.), 0.4 ng/ml visfatin (Biovision, Mountain view, Calif.) and amino acids (Invitrogen) at final concentrations described in Table 1. This cocktail promoted in vitro microsteatosis reduction by activating hepatocellular TG metabolism. SRS medium pH was adjusted to match that of NSRS. Cells were incubated in SRS or NSRS medium for 48 hours, after which (13 days post-seeding) the spent medium from all cultures was collected and replaced with NSRS medium for another 48 hours. On post-seeding day 15, the spent medium from all experimental conditions was collected (FIG. 5A).

TABLE 1
Amino acid (mg/L) concentrations supplemented to
steatosis reducing culture medium, SRS.

| Amino acids | Concentration (mg/L) |
|---|---|
| L-Arginine hydrochloride | 252.8 |
| L-Cystine | 48 |
| L-Histidine hydrochloride-H2O | 84 |
| L-Isoleucine | 104.8 |
| L-Leucine | 104.8 |
| L-Lysine hydrochloride | 145 |
| L-Methionine | 30.2 |
| L-Phenylalanine | 66 |
| L-Threonine | 95.2 |
| L-Tryptophan | 20.4 |
| L-Tyrosine | 72 |
| L-Valine | 93.6 |
| Glycine | 30 |
| L-Alanine | 35.6 |
| L-Asparagine | 52.8 |
| L-Aspartic acid | 53.2 |
| L-Glutamic Acid | 58.8 |
| L-Proline | 46 |
| L-Serine | 42 |
| L-Glutamine | 584 |

Note:
these concentrations reflect only amino acid concentrations due to supplementation.

### Hepatocyte Steatosis Assessment

A nondestructive quantitative image analysis method was used to quantify lipid droplet size distribution. Hepatocyte cultures were fixed in 4% paraformaldehyde, stained with the lipid-specific Nile red stain (Adipored™, Lonza, Walkersville, Md.), and counterstained with 1 μg/ml nuclei-specific Hoechst-33342 stain (Invitrogen), following the manufacturer recommendations. Confocal fluorescence images were obtained with an Olympus IX-80 microscope and analyzed using the product disclosed here, yielding unbiased measurements of size (cross-sectional surface area) and lipid droplet distribution/cell (FIGS. 3-5).

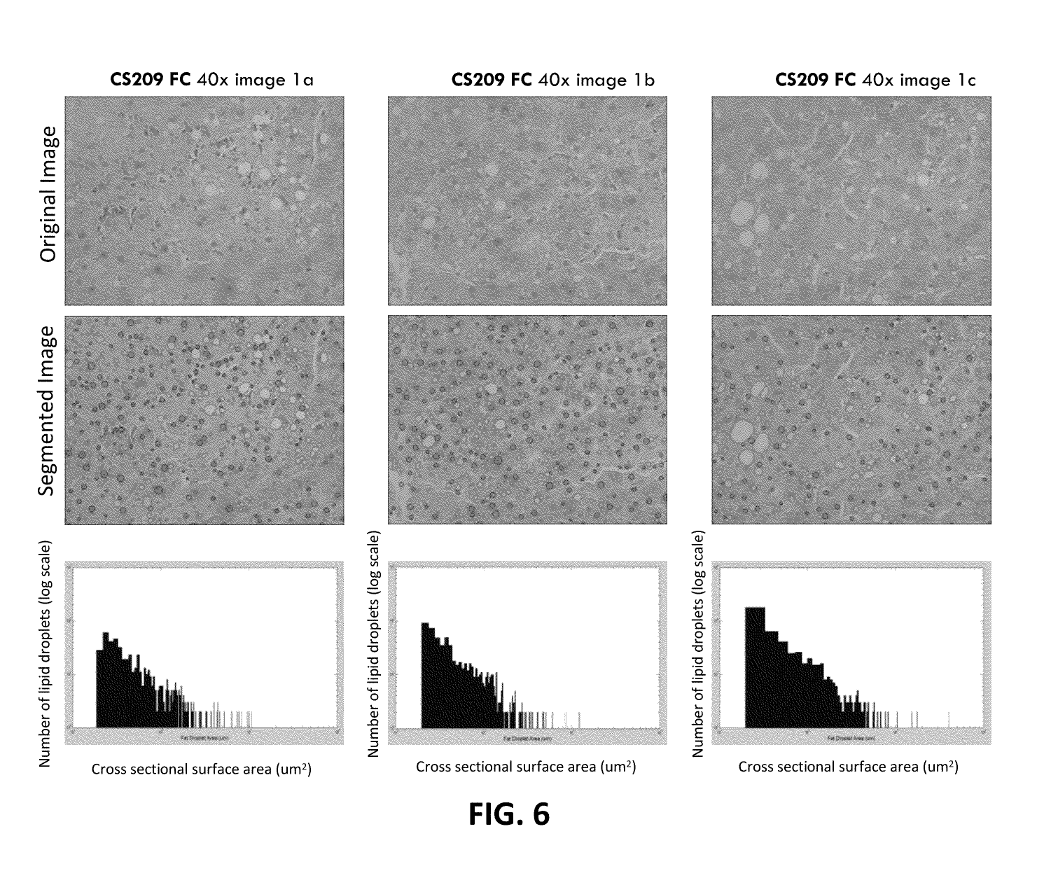

Human liver tissue samples were obtained, stained with hematoxylin and eosin (H&E) and imaged as described in the literature (Guarrera et al., J. Surg. Res., 2011, 167: e365-e373). For example, FIG. 6 shows a histogram representing count of fat droplets vs. size of fat droplets.

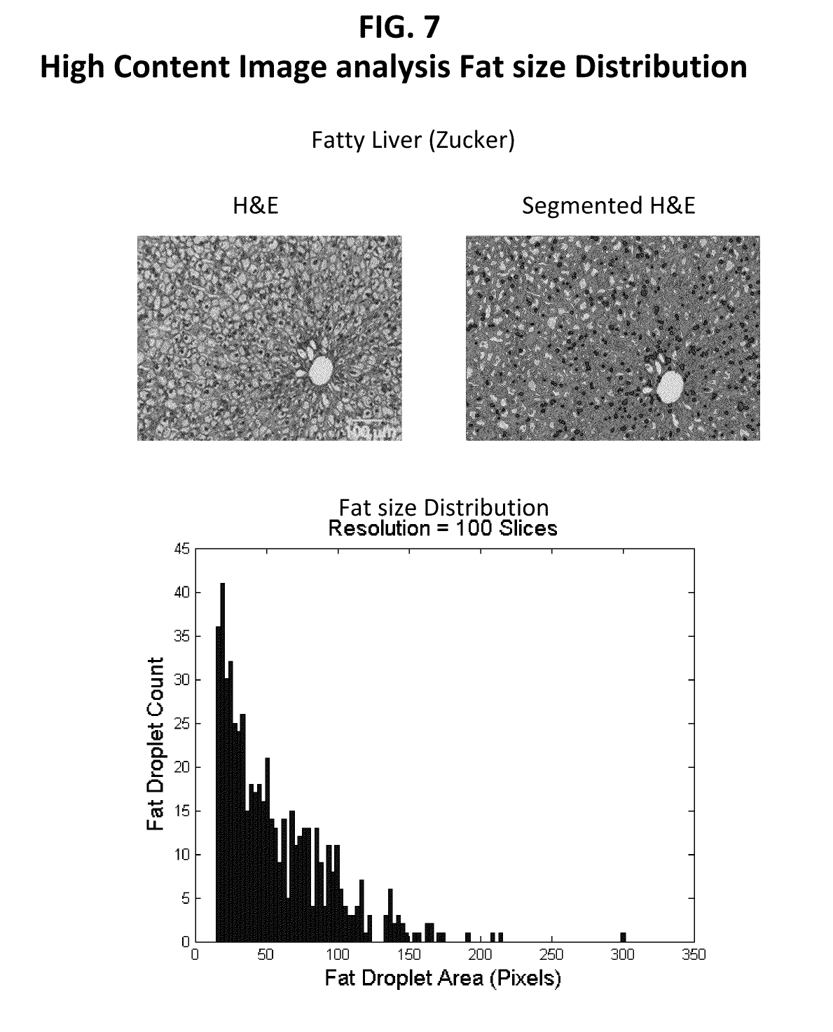

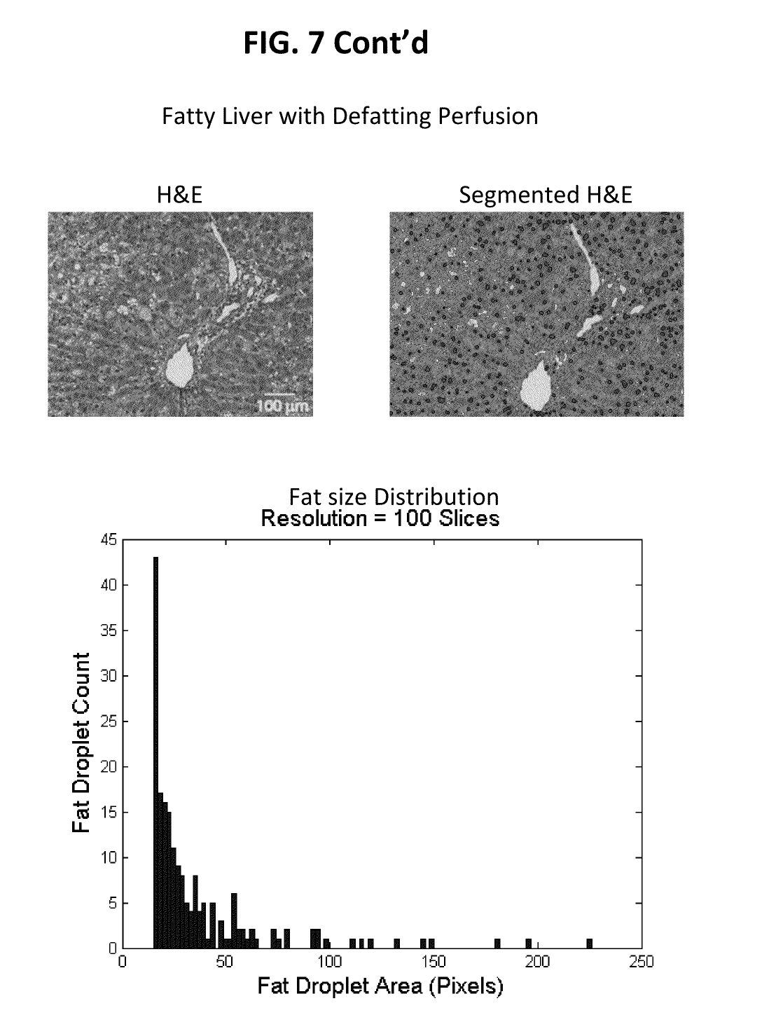

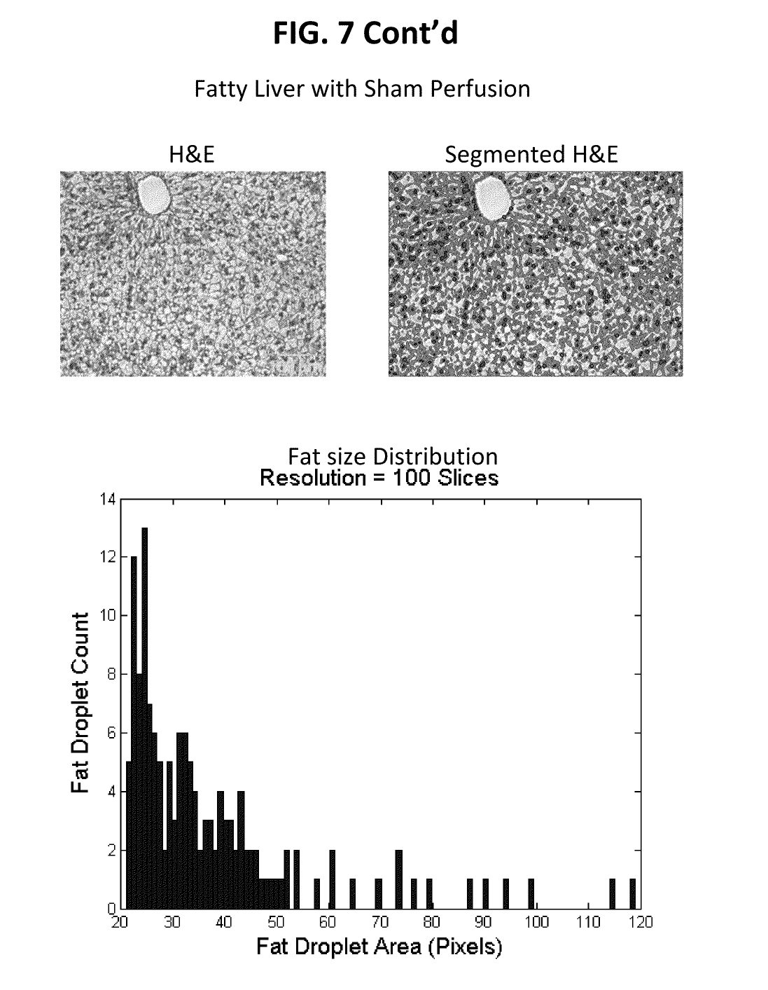

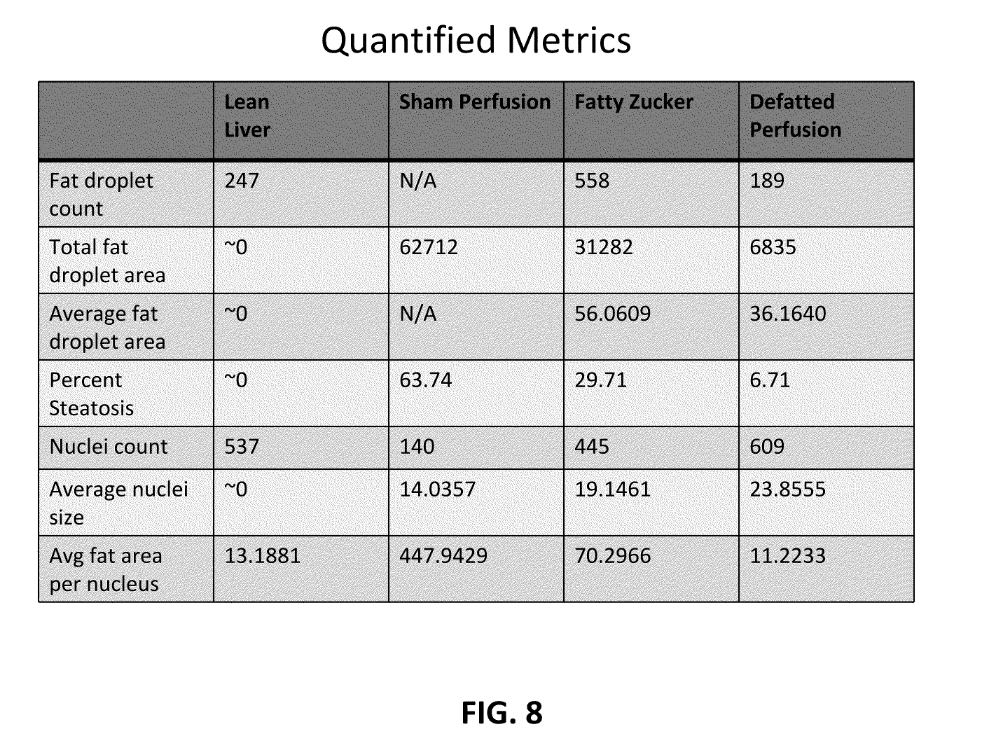

The product of the invention was used to perform an automated image analysis on digital images of liver histology slides stained with H&E stain. These images are disclosed by Nagrath et al., Metab. Eng., 2009, 11, 274-283, which is hereby incorporated by reference in its entirety. Using the product of the present invention, the size distribution of fat droplets in rat livers at different steatosis levels was accurately quantified. Results are shown in FIGS. 7-8. The results were in agreement with the qualitative analysis of a certified pathologist as indicated in Nagrath et al.

The foregoing examples and description of the preferred embodiments should be taken as illustrating, rather than as limiting, the present invention as defined by the claims. As will be readily appreciated, numerous variations and combinations of the features set forth above can be utilized without departing from the present invention as set forth in the claims. Such variations are not regarded as a departure from the spirit and script of the invention, and all such variations are intended to be included within the scope of the following claims. All reference cited herein are incorporated by reference in their entireties.

What is claimed is:

1. A method for assessing the state of steatosis of a tissue, the method comprising: (a) providing a sample of the tissue; (b) conducting an automated image analysis on a digital image of the sample tissue or cells thereof; (c) generating an array of parameters related to steatosis; and determining intra-cellular triglyceride content in the form of lipid droplets in the tissue based upon the parameters.

2. The method of claim 1, wherein said array of parameters are independently selected from the group consisting of fat droplet count, total fat droplet cross-sectional surface area, average fat droplet cross-sectional surface area, average lipid droplet equivalent diameter, total area percent steatosis, nuclei count, total nuclei area, average nucleus area, nuclei proximity to a lipid droplet, and average fat area per nucleus.

3. The method of claim 1, wherein said automated analysis comprises the steps of: 1) uploading digital images, 2) analyzing high content images, 3) displaying results in the forms of images, tables or histograms, 4) outputting the analysis results to external formats, and 5) predicting state of liver steatosis based on machine learning.

4. The method of claim 1, further comprising using an algorithm to automatedly determine the level of steatosis of any given tissue slice or culture system, incorporating a plurality of methods of defining steatosis levels from which a user can select the preferred modality.

5. The method of claim 4, wherein the algorithm comprises a metric that finds the ratio of total fat area to total tissue area, or total macrosteatotic area to total tissue area.

6. The method of claim 4, comprising defining steatosis as the number of macrosteatotic droplets in the slice divided by the number of cell nuclei in the slice.

7. The method of claim 4, combining the macrosteatotic area metric with the macro count metric and using a latent classification metric to determine percent steatosis in the tissue.

8. An automated high-content image analysis system for assessing intracellular fat content of a tissue, comprising: (a) a software program comprising a plurality of object-oriented module-based algorithms capable of segmenting intracellular fat droplets, cell nuclei, sinusoidal spaces, erythrocytes, and other cellular structures; (b) a clustering mechanism capable of roughly separating fat, nuclei, and surrounding tissue before more sensitive, model-driven approaches to tune the classification schemes toward maximal accuracy; (c) an algorithm capable of generating Edge characteristics using the Laplacian of Gaussians technique; (d) an active contour or level set algorithm; and (e) a graph partitioning mechanism.

9. The automated high-content image analysis system of claim 8, further comprising a multi-threshold approach in combination with shape, intensity, texture, and edge based descriptors to differentiate between different components of tissue.

10. The automated high-content image analysis system of claim 8, wherein said Edge characteristics comprise steep changes in intensity characteristic of object boundaries and a Watershed filter to refine geometrical segmentation criteria.

11. The automated high-content image analysis system of claim 8, wherein said graph partitioning is employed during the multithreshold and level set routines as an overarching framework to track the progression of geometrical change in response to changing thresholds and sets.

12. The automated high-content image analysis system of claim 11, wherein in the case of said multithreshold approach, as the stringency of the highpass filter is iteratively increased, the number of structures that pass the threshold will decrease, and will subserve those structures that passed the previous threshold; and wherein weighted directed graphs are used to track these relationships.

13. A computer readable medium having instructions for enabling a computer system to implement the method of claim 1.

14. A method of identifying agents for reducing triglyceride content in the form of lipid droplets in a liver in need of such reduction, comprising: (1) obtaining a tissue sample from a fatty liver or from lean liver induced for steatosis in vitro; (2) contacting said tissue sample with one or more candidate agents; and (3) subjecting said tissue sample to an automated image analysis system according to claim 8 to determine the state of steatosis in the sample, wherein a reduction in the state of steatosis in the sample contacted with a candidate agent relative to an untreated control sample is indicative of the ability of the candidate agent to reduce the triglyceride content in a fatty liver.

15. A device for assessing intra-cellular triglyceride content in the form of lipid droplets in a liver tissue for the state of steatosis, comprising an automated high-content image analysis system of claim 8.

16. A machine learning framework for determining the sensitivity and specificity of the automated high-content image analysis system of claim 8 in comparison with analysis by a clinician, comprising bootstrapping, rooted trees, Bayesian clustering, graphical partitioning, and a plurality of forms of component analysis (PCA, ICA, etc.), wherein the methods provide a way for the program to automatedly learn based on a small portion of the full dataset that is used as a training set.

17. The machine learning framework of claim 16, wherein the program is able to a) determine the inputs that maximize accuracy and stability; and b) reduce the number of inputs that the clinician needs to define, thereby becoming more automated given more data.

18. An automated classifier for segregating macrosteatotic fat droplets from microsteatotic fat droplets, based on the propensity of a given sized droplet to cause a nucleus displacement toward the side of a cell, wherein macrosteatotic droplets cause a positional shift to the cell nucleus, pushing them toward the side of the cell, wherein the nuclei remain in a state such that they are directly adjacent to the macrosteatotic droplet.

19. The automated classifier of claim 18, comprising a software program that uses a “nucleus adjacency” probability metric to define a size-based cutoff that categorizes macro and micro droplets.

20. The automated classifier of claim 19, wherein the software linearly combines the nucleus adjacency score and the size score to form a latent score that classifies macro and micro steatosis.
